(19) United States
(12) Patent Application Publication
(10) Pub. No.: US 2026/0335671 A1
(43) Pub. Date: Oct. 29, 2026

(54) SYSTEM AND METHOD FOR LIGHT ACTIVATED DERMATOLOGIC, COSMETIC, OR MEDICAL TREATMENT USING SURFACE PLASMON RESONANCE TUNED GOLD NANORODS

(71) Applicants: Christian Schoen, Loveland, CO (US); Maximilian Schoen, Ft Collins, CO (US); Christopher Sievers, Greeley, CO (US)

(72) Inventors: Christian Schoen, Loveland, CO (US); Maximilian Schoen, Ft Collins, CO (US); Christopher Sievers, Greeley, CO (US)

(21) Appl. No.: 19/545,901

(22) Filed: Feb. 20, 2026

Related U.S. Application Data
(60) Provisional application No. 63/761,390, filed on Feb. 21, 2025.
(60) Provisional application No. 63/769,063, filed on Mar. 9, 2025.
(60) Provisional application No. 63/839,243, filed on Jul. 6, 2025.
(60) Provisional application No. 63/839,164, filed on Jul. 6, 2025.
(60) Provisional application No. 63/870,233, filed on Aug. 26, 2025.
(60) Provisional application No. 63/880,790, filed on Sep. 12, 2025.

(51) Int. Cl. A61N 5/06; A61M 37/00

(52) U.S. Cl. CPC A61N 5/062; A61M 37/0015; A61N 5/0616; A61M 2037/0023; A61M 2037/0046; A61N 2005/0651; A61N 2005/0659; A61N 2005/0663

(57) ABSTRACT

A dermatologic, cosmetic, or medical treatment system utilizing surface plasmon resonance-tuned gold nanorods activated by light is disclosed. The system includes a topical composition and a light-emitting device configured for spectral overlap activation. Mechanical and ligand-functionalized embodiments provide enhanced delivery and activation options.

### CROSS-REFERENCE TO RELATED APPLICATIONS

This application claims priority to U.S. Provisional Application Nos. 63/761,390; 63/769,063; 63/839,243; 63/839,164; 63/870,233; and 63/880,790.

### FIELD OF THE INVENTION

The invention relates to dermatologic, cosmetic, and medical treatment systems using light-activated surface plasmon resonance-tuned gold nanorods.

### BACKGROUND

Light-based dermatologic devices and plasmonic nanomaterials are known independently. Integrated systems that coordinate spectral overlap between light sources and nanorods provide enhanced activation efficiency and improved therapeutic or cosmetic performance.

### SUMMARY

The invention provides layered system, method, mechanical delivery, and ligand-functionalized embodiments enabling photothermal and photobiomodulatory activation in epidermal or dermal tissue.

### DETAILED DESCRIPTION

In various embodiments, gold nanorods exhibit a longitudinal surface plasmon resonance (LSPR) peak tunable between approximately 500 nm and 1200 nm. Nanorods may have lengths between 10-200 nm, diameters between 5-50 nm, and aspect ratios between 2:1 and 15:1. Concentrations may range from trace amounts to several weight percent within dermatologically acceptable carriers including creams, gels, lotions, serums, hydrogels, emulsions, or ointments. Light sources may include LEDs, diode lasers, solid-state lasers, fiber lasers, or IPL systems emitting wavelengths between 400-1550 nm. Spectral overlap between emission spectrum and LSPR peak enhances activation efficiency. Power densities and exposure durations may be adjusted to achieve photothermal or photobiomodulatory modulation. Mechanical applicators may include roller balls, microneedle arrays (solid or dissolvable), vibratory heads, or sonic devices. Nanorods may be functionalized with peptides, antioxidants, retinoids, growth factors, or thermally/light-responsive ligands enabling controlled activation or release.

1. A dermatologic, cosmetic, or medical treatment system comprising:
a) a topical composition comprising surface plasmon resonance-tuned gold nanorods; and
b) a light-emitting device configured to emit light suitable for activating the gold nanorods;
wherein illumination induces localized photothermal or photobiomodulatory effects in tissue.

2. The system of claim 1, wherein the emitted wavelength overlaps a longitudinal surface plasmon resonance peak of the gold nanorods.

3. The system of claim 1, wherein the nanorods exhibit a longitudinal surface plasmon resonance peak between 500 nm and 1200 nm.

4. The system of claim 1, wherein the nanorods have an aspect ratio between 2:1 and 15:1.

5. The system of claim 1, wherein the nanorods have a length between 10 nm and 200 nm.

6. The system of claim 1, wherein the nanorods have a diameter between 5 nm and 50 nm.

7. The system of claim 1, wherein the light-emitting device emits light between 400 nm and 1550 nm.

8. The system of claim 1, wherein the light-emitting device comprises LEDs.

9. The system of claim 1, wherein the topical composition further comprises at least one cosmetic or therapeutic active.

10. The system of claim 9, wherein the active is selected from peptides, antioxidants, vitamins, retinoids, or growth factors.

11. A method comprising applying a topical composition comprising surface plasmon resonance-tuned gold nanorods to tissue and exposing the tissue to activating light.

12. The method of claim 11, wherein the light overlaps the longitudinal surface plasmon resonance peak of the nanorods.

13. The method of claim 11, wherein exposure duration is between seconds and minutes.

14. The method of claim 11, wherein the method enhances collagen production or improves tissue appearance.

15. A system comprising a topical composition comprising surface plasmon resonance-tuned gold nanorods and a mechanical applicator selected from roller ball, microneedle roller, vibrating head, sonic wand, or massaging applicator.

16. The system of claim 15, wherein the applicator comprises a microneedle array.

17. The system of claim 16, wherein the microneedle array is dissolvable.

18. The system of claim 15, further comprising a light-emitting device configured to activate the nanorods after mechanical positioning.

19. A method comprising applying nanorods functionalized with a light-responsive ligand and exposing the nanorods to activating light.

20. The method of claim 19, wherein activation induces ligand release.

21. The method of claim 19, wherein the ligand is thermally or photo-responsive.
